(19) United States
(12) Patent Application Publication
(10) Pub. No.: US 2004/0030167 A1
(43) Pub. Date: Feb. 12, 2004

(54) 3-Methyl-20-epi-vitamin d derivatives

(76) Inventors: Hiroaki Takayama, Tokyo (JP); Toshie Fujishima, Tokyo (JP)

Correspondence Address: BIRCH STEWART KOLASCH & BIRCH, PO BOX 747, FALLS CHURCH, VA, 22040-0747, US

(21) Appl. No.: 10/332,124

(22) PCT Filed: Mar. 2, 2001
(86) PCT No.: PCT/JP01/01641

(30) Foreign Application Priority Data
Aug. 8, 2000 (JP) 239799/2000

(51) Int. Cl. C07C 401/00; A61K 31/59

(52) U.S. Cl. 552/653; 514/167

(57) ABSTRACT

The object of the present invention is to synthesize vitamin D derivatives in which the 3-position is substituted with methyl and the steric configuration at the 20-position is epimerized.

The present invention provides vitamin D derivatives of Formula (1):

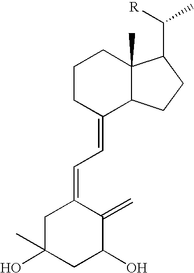

wherein R is straight or branched alkyl optionally substituted with hydroxy.

### TECHNICAL FIELD

The present invention relates to novel vitamin D derivatives, more particularly, relates to 3-methyl-20-epi-vitamin D derivatives, in which the steric configuration at the 20-position is not native and the 3-position is substituted with methyl.

### BACKGROUND ART

Vitamin D derivatives including 1α,25-dihydroxyvitamin D₃ are known to have many physiological activities such as calcium metabolism regulatory activities, growth inhibitory and differentiation inducing activities for tumor cells and immunoregulatory activities. However, some active vitamin D₃ derivatives may cause hypercalcemia during long-term and continuous administration, therefore they are not suitable for use as antitumor agents, antirheumatic agents and the like. Thus, a number of synthetic studies have been conducted to obtain such vitamin D derivatives that are excellent in specific activities among the above-mentioned activities.

For example, if the A-ring of an active vitamin D₃ derivative is substituted, the possible conformation of the molecule may be limited, resulting in a characteristic activity of the resulting vitamin D derivative. For example, 1α,25-dihydroxyvitamin D₃ derivatives having methyl at the 2- or 4-position are described by K. Konno et al. (Bioorg. Med. Chem. Lett., 1998, 8, 151) and T. Fujishima et al. (ibid., 1998, 8, 2145) and in Abstracts of the 118th Annual Meeting of the Pharmaceutical Society of Japan 2 (p.171). In addition, a vitamin D derivative having methyl at the 3-position is described in Abstracts of the 120th Annual Meeting of the Pharmaceutical Society of Japan 2 (p.105). However, no vitamin D₃ derivative has been reported in which the 3-position is substituted with methyl and the steric configuration at the 20-position is epimerized.

### DISCLOSURE OF THE INVENTION

An object of the present invention is to provide and to synthesize 3-methyl-20-epi-vitamin D derivatives. Another object of the present invention is to evaluate biological activity of the resulting 3-methyl-20-epi-vitamin D derivatives.

As a result of careful studies so as to achieve the above mentioned objects, the inventors of the present invention have succeeded in synthesizing desired vitamin D derivatives by coupling A-ring part precursors and CD-ring parts using palladium catalyst after synthesizing the A-ring part precursors and the CD-ring parts separately by the method described in Abstracts of the 120th Annual Meeting of the Pharmaceutical Society of Japan 2 (p.105) and by the method described by T. Fujishima et al. (Bioorg. Med. Chem., 2000, 8, 123), respectively; thereby they achieved the present invention.

According to one aspect of the present invention, there is provided a vitamin D derivative of Formula (1):

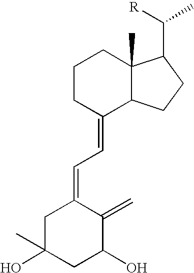

wherein R is straight or branched alkyl optionally substituted with hydroxy.

For R of Formula (1), straight or branched C₁₋₁₂ alkyl substituted with hydroxy is preferred and straight or branched C₁₋₁₀ alkyl substituted with hydroxy is more preferred.

Particularly preferably, R is 4-hydroxy-4-methylpentyl or 4-ethyl-4-hydroxyhexyl, more preferably R is 4-hydroxy-4-methylpentyl.

According to another aspect of the present invention, there is provided the use of the vitamin D derivative of the present invention for the preparation of a therapeutic agent for diseases associated with calcium metabolic disorder.

According to yet another aspect of the present invention, there is provided a method of treating a disease associated with calcium metabolic disorder, which method comprises a step of administering a therapeutically effective amount of the vitamin D derivative of the present invention to a patient in need of such treatment.

The vitamin D derivative of the present invention can be used as a test reagent in studying the metabolism of active vitamin D₃ (i.e., 1α,25-dihydroxyvitamin D₃).

### PREFERRED MODE FOR CARRYING OUT THE INVENTION

The contents of the specification of Japanese Patent Application No. 2000-239799, the application on the basis of which the present application claims priority are to be incorporated in their entirety by reference.

Detailed modes and methods with respect to vitamin D derivatives of Formula (1) of the present invention are described in further detail below.

In the present specification, “straight or branched alkyl” is preferably straight or branched C₁₋₁₅ alkyl; examples thereof include methyl, ethyl, n-propyl, i-propyl, n-butyl, s-butyl, i-butyl and t-butyl, and further include pentyl, hexyl, heptyl, octyl, nonyl, decanyl, etc.

“Straight or branched alkyl optionally substituted with hydroxy” means that one or more hydrogen atoms of the above-mentioned straight or branched alkyl may be substituted with hydroxy. In the definition of R, the number of hydrogen atoms substituted with hydroxy is preferably 1, 2 or 3, more preferably 1 or 2 and most preferably 1.

Preferably R is straight or branched C₁₋₁₂ alkyl substituted with hydroxy, more preferably straight or branched C₃₋₁₀ alkyl substituted with hydroxy. Non-limiting examples of R include 4-hydroxy-4-methylpentyl, 4-ethyl-4-hydroxyhexyl, 6-hydroxy-6-methyl-2-heptyl, 7-hydroxy-7-methyl-2-octyl, 5,6-dihydroxy-6-methyl-2-heptyl, 4,6,7-trihydroxy-6-methyl-2-heptyl, etc. More preferably R is 4-hydroxy-4-methylpentyl or 4-ethyl-4-hydroxyhexyl and most preferably R is 4-hydroxy-4-methylpentyl.

The vitamin D derivatives of Formula (1) of the present invention can be used as active ingredients of pharmaceutical compositions (such as a calcium metabolism regulating agent).

Although there is no limitation with respect to methods of synthesizing vitamin D derivatives of Formula (I) of the present invention which are novel compounds, they can be synthesized, for example, by synthesizing A-ring and CD-ring parts of the vitamin D derivatives separately and then coupling them together, as described in the following Examples.

CD-ring part compounds of vitamin D derivatives are known. Alternatively a desired CD-ring compound is obtainable by appropriately modifying a side chain of a known CD-ring compound or is obtainable from a known vitamin D derivative having a corresponding side chain.

Examples of such a known vitamin D derivative include those which are disclosed in Japanese Patent Publication (Kokai) Nos. 61-267550 A, 6-72994 A and 6-256300 A and Japanese Patent Publication (Kohyo) Nos. 4-503669 A, 4-504573 A and 10-182597 A, WO94/14766, WO95/27697, etc.

According to Scheme 4 described by T. Fujishima et al (Bioorg. Med. Chem., 2000, 8, 123), a CD-ring compound having a desired side chain is obtainable as follows: an aldehyde led from the ozonolysis of vitamin D₂ is treated with a base to epimerize the stereochemistry on a carbon, the position of which corresponds to the 20-position of the steroid skeleton. A desired side chain is introduced to the epimerized aldehyde to give a protected alcohol, which is then deprotected and oxidized. Thus obtained ketone is converted to a bromomethylene to give a CD-ring compound having the desired side chain.

An A-ring compound having methyl at the 3-position is synthesizable by the method described on page 105 of Abstracts of the 120th Annual Meeting of the Pharmaceutical Society of Japan 2 via a 3-methylbutane-1,2,4-triol derivative, which is synthesizable from 3-methylbut-3-en-1-ol, as a starting material; however there is no limitation with respect to a method for synthesizing the compounds.

An A-ring compound and a CD-ring compound can be coupled by a known conventional method. Namely, an A-ring compound, which is obtainable by the above method and which has a triple bond at one terminal and a double bond at the other terminal, is reacted with a CD-ring compound, which has bromomethylene at the coupling site for the A-ring compound, in the presence of a palladium catalyst in an appropriate solvent.

After the coupling reaction, the resulting product is purified in a usual manner such as thin layer chromatography and subjected to removal of the hydroxy protecting groups, to give a desired vitamin D derivative.

The present invention will be described specifically by way of the following Examples, which in no way limit the invention. The following schemes show the reactions carried out in Examples.

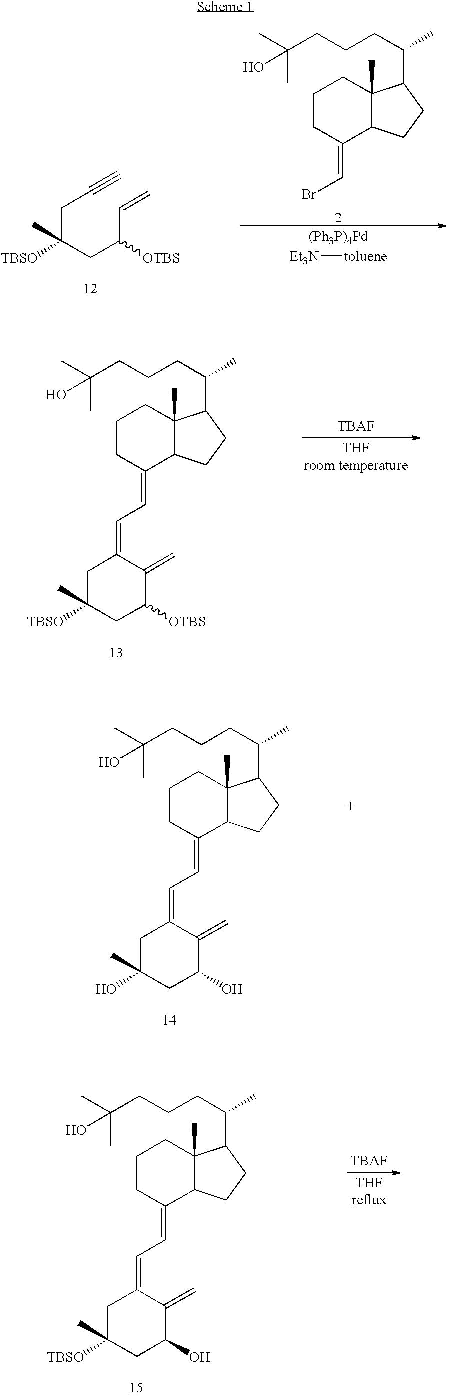

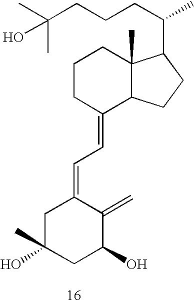

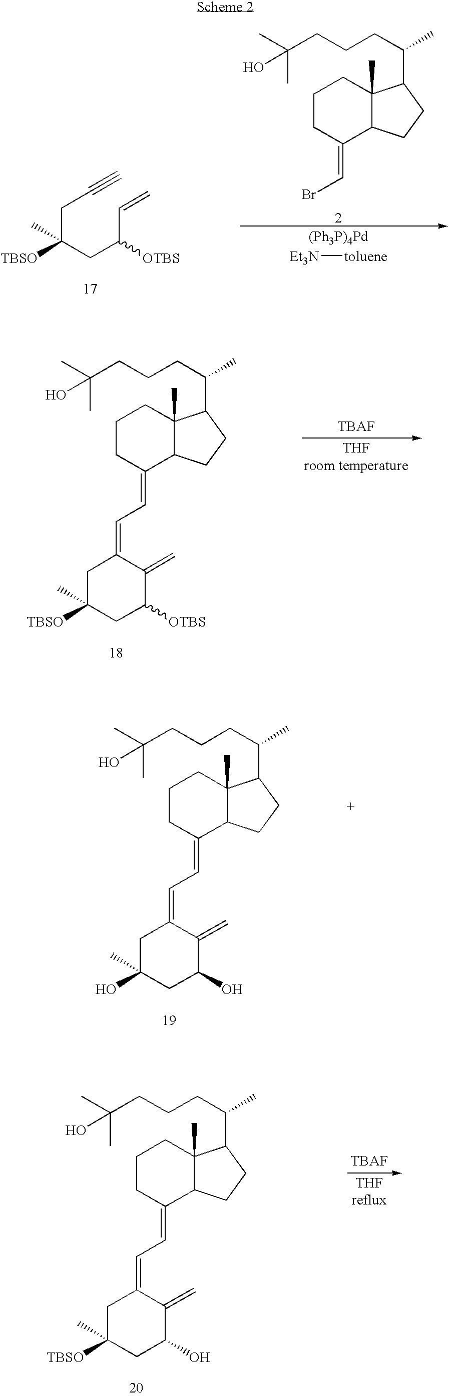

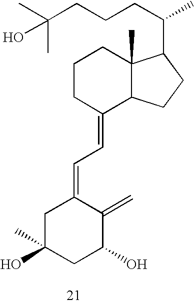

### EXAMPLES

The present invention will be described specifically by way of the following Examples, which in no way limit the invention.

### Example 1

### Synthesis of (5Z,7E)-(1R,3R,20S)-3-methyl-9,10-seco-5,7,10(19)-cholestatriene-1,3,25-triol (Compound 14)

(E)-de-A,B-8-(bromomethylene)cholestan-25-ol (Compound 2) (124 mg, 0.35 mmol) and triethylamine (5 ml) were mixed in toluene (3 ml); the resulting solution was mixed with (Ph₃P)₄Pd (121 mg, 0.11 mmol) and stirred at room temperature for 10 minutes. A solution of an A-ring compound (Compound 12) (64 mg, 0.18 mmol) in toluene (2 ml) was then added, followed by stirring at room temperature for a further 10 minutes. The A-ring Compound 12 was synthesized by the method described on page 105 of Abstracts of the 120th Annual Meeting of the Pharmaceutical Society of Japan 2 via a 3-methylbutane-1,2,4-triol derivative, which had been synthesized from 3-methylbut-3-en-1-ol, as a starting material. The reaction mixture was heated under reflux for 1.5 hours, mixed with brine and extracted with ethyl acetate. The thus obtained organic layer was dried over magnesium sulfate and filtered. The filtrate was concentrated. Thus obtained crude product was purified by silica gel chromatography (ethyl acetate:n-hexane=1:10) to give Compound 13 as a colorless oil (151 mg, 66% yield).

The above mentioned Compound 13 (68 mg, 0.10 mmol) which was a protected vitamin D derivative was dissolved in THF (2 ml). While stirring thus obtained solution at 0° C. under an argon atmosphere, TBAF (tetrabutylammonium fluoride) (1.0 M solution in THF, 0.3 ml, 0.3 mmol) was added. The reaction mixture was stirred at room temperature for 6 hours and 45 minutes, mixed with brine and extracted with ethyl acetate. The organic layer was dried over magnesium sulfate and filtered. The filtrate was evaporated to remove the solvent and thus obtained crude product was purified by silica gel chromatography (ethyl acetate:n-hexane=1:5 and then 1:1) to give Compound 14 (16 mg, 41%) and Compound 15 (23 mg, 44%), each as a white solid. Compound 14 was further purified by reverse phase recycle HPLC (YMC-Pack ODS column, 20 mm×150 mm, 9.0 ml/min, acetonitrile:water=8:2) for biological activity evaluation.

UV (EtOH) λmax 266 nm, λmin 226 nm; ¹H NMR (400 MHz, CDCl₃) δ 0.55 (3 H, s), 0.85 (3H, d, J=6.4 Hz), 1.19 (6 H, s), 1.23 (3 H, s), 1.80 (1 H, dd, J=14.3, 3.4 Hz), 2.11 (1 H, ddd, J=14.3, 3.1, 2.4 Hz), 2.40 (2H, m), 2.84 (1 H, dd, J=11.3, 3.7 Hz), 4.42 (1 H, t, J=3.1 Hz), 5.02 (1 H, d, J=2.1 Hz), 5.28 (1 H, d, J=2.1 Hz), 6.07 (1 H, d, J=11.3 Hz), 6.45 (1 H, d, J=11.3 Hz); MS 430 [M]⁺, 412 [M−H₂O]⁺; HRMS calcd. for [C₂₈H₄₆O₃] 430.3447, found 430.3448.

### Example 2

### Synthesis of (5Z,7E)-(1S,3R,20S)-3-methyl-9,10-seco-5,7,10(19)-cholestatriene-1,3,25-triol (Compound 16)

Under an argon atmosphere at 60° C., a solution of Compound 15 (23 mg, 0.041 mmol) in THF (1 ml) was treated with TBAF (1.0 M solution in THF, 0.4 ml, 0.4 mmol) for 15 hours. After the treatment, brine was added to thus obtained mixture and extracted with ethyl acetate. The organic layer was dried over magnesium sulfate and filtered. The filtrate was evaporated for removing the solvent to give a crude product. This crude product was subjected to silica gel chromatography (ethyl acetate:n-hexane=2:1) to give Compound 16 (17 mg) as a white solid. The yield was 82%. Compound 16 was further purified by reverse phase recycle HPLC (YMC-Pack ODS column, 20 mm×150 mm, 9.0 ml/min, acetonitrile:water=8:2) for biological activity evaluation.

UV (EtOH) λmax 263 nm, λmin 228 nm; ¹H NMR (400 MHz, CDCl₃) δ 0.55 (3 H, s), 0.85 (3H, d, J=6.4 Hz), 1.21 (6 H, s), 1.32 (3 H, s), 1.51 (1 H, dd, J=12.2, 11.6 Hz), 2.19 (1 H, ddd, J=12.8, 5.5, 2.7 Hz), 2.24 (1 H, dd, J=14.0, 2.4 Hz), 2.42 (1 H, d, J=13.7 Hz), 2.81 (1 H, m), 4.34 (1 H, ddt, J=11.3, 5.2, 2.2 Hz), 5.02 (1 H, t, J=1.8 Hz), 5.38 (1 H, t, J=1.8 Hz), 6.05 (1 H, d, J=11.6 Hz), 6.32 (1 H, dd, J=11.0, 1.8 Hz); MS 430 [M]⁺, 412 [M−H₂O]⁺; HRMS calcd. for [C₂₈H₄₆O₃] 430.3447, found 430.3447.

### Example 3

### Synthesis of (5Z,7E)-(1S,3S,20S)-3-methyl-9,10-seco-5,7,10(19)-cholestatriene-1,3,25-triol (Compound 19)

(E)-de-A,B-8-(bromomethylene)cholestan-25-ol (Compound 2) (237 mg, 0.62 mmol) and triethylamine (5 ml) were dissolved in toluene (6 ml), to which (Ph₃P)₄Pd (104 mg, 0.09 mmol) was added and stirred at room temperature for 10 minutes. Then, a solution of Compound 17 (110 mg, 0.31 mmol) which was an A-ring part compound in toluene (2 ml) was added to the mixture, followed by stirring for a further 10 minutes at room temperature. Compound 17, an A-ring part, was synthesized by the method described in Abstracts of the 120th Annual Meeting of the Pharmaceutical Society of Japan 2 (p.105) via a 3-methylbutane-1,2,4-triol derivative, which was synthesized from 3-methylbut-3-en-1-ol, as a starting material. The mixture was heated under reflux for 1.5 hours, mixed with brine and extracted with ethyl acetate. The organic layer was dried over magnesium sulfate and filtered. The filtrate was concentrated. The thus obtained crude product was purified by silica gel chromatography (ethyl acetate:n-hexane=1:9) to give Compound 18 (171 mg) as a colorless oil. The yield was 85%.

TBAF (1.0 M solution in THF, 0.77 ml, 0.77 mmol) was added to a stirred solution of the above mentioned Compound 18 (169 mg, 0.26 mmol) in THF (3.5 ml) under an argon atmosphere at 0° C. The reaction mixture was stirred at room temperature for 7 hours, mixed with brine, and extracted with ethyl acetate. The organic layer was dried over magnesium sulfate and filtered. The filtrate was evaporated to remove the solvent and thus obtained crude product was purified by silica gel chromatography (ethyl acetate:n-hexane=1:5 and then 1:1) to give Compound 19 (29 mg, 21%) and Compound 20 (54 mg, 49%), each as a white solid. Compound 19 was further purified by reverse phase recycle HPLC (YMC-Pack ODS column, 20 mm×150 mm, 9.0 ml/min, acetonitrile:water=8:2) for biological activity evaluation.

UV (EtOH) λmax 265 nm, λmin 227 nm; ¹H NMR (400 MHz, CDCl₃) δ 0.53 (3 H, s), 0.84 (3 H, d, J=6.4 Hz), 1.21 (6 H, s), 1.32 (3 H, s), 1.81 (1 H, dd, J=14.4, 3.4 Hz), 2.09 (1 H, m), 2.40 (2 H, m), 2.84 (1 H, dd, J=11.9, 4.0 Hz), 4.39 (1 H, t, J=3.1 Hz), 4.98 (1 H, d, J=1.8 Hz), 5.26 (1 H, d, J=1.8 Hz), 6.02 (1 H, d, J=11.3 Hz), 6.44 (1 H, d, J=11.0 Hz); MS 430 [M]⁺, 412 [M−H₂O]⁺, HRMS calcd. for [C₂₈H₄₆O₃] 430.3447, found 430.3465.

### Example 4

### Synthesis of (5Z,7E)-(1R,3S,20S)-3-Methyl-9,10-seco-5,7,10(19)-cholestatriene-1,3,25-triol (Compound 21)

Under an argon atmosphere at 60° C., a solution of Compound 20 (29 mg, 0.053 mmol) in THF (1.5 ml) was treated with TBAF (1.0 M solution in THF, 0.5 ml, 0.5 mmol) for 15 hours. After the treatment, brine was added to thus obtained mixture and extracted with ethyl acetate. The organic layer was dried over magnesium sulfate and filtered. The filtrate was evaporated for removing the solvent to give a crude product, which was then subjected to silica gel chromatography (ethyl acetate:n-hexane=2:1) to give Compound 21 (17 mg) as a white solid. The yield was 74%. Compound 21 was further purified by reverse phase recycle HPLC (YMC-Pack ODS column, 0 mm×150 mm, 9.0 ml/min, acetonitrile:water=8:2) for biological activity evaluation.

UV (EtOH) λmax 263 nm λmin 228 nm; ¹H NMR (400 MHz, CDCl₃) δ 0.53 (3 H, s), 0.85 (3H, d, J=6.7 Hz), 1.21 (6 H, s), 1.30 (3 H. s), 1.53 (1 H, dd, J=12.5, 11.0 Hz), 2.18 (1 H, ddd, J=12.5, 5.2, 2.4 Hz), 2.25 (1 H, dd, J=13.7, 2.1 Hz), 2.41 (1 H, d, J=13.7 Hz), 2.81 (1 H, dd, J=12.2, 4.0 Hz), 4.36 (1 H, ddt, J=11.0, 5.2, 1.8 Hz), 4.97 (1 H, m), 5.36 (1 H, t, J=1.8 Hz), 6.10 (1 H, d, J=11.3 Hz), 6.32 (1 H, dd, J=11.3 Hz); MS 430 [M]⁺, 412 [M−H₂O]⁺; HRMS calcd. for [C₂₈H₄₆O₃] 430.3447, found 430.3444.

### Test Example

### Assay for Binding to Bovine Thymus Vitamin D Receptor (VDR)

Compounds 14, 16, 19 and 21 synthesized in the above Examples 1 to 4 were tested for their binding properties to bovine thymus vitamin D receptor.

Ethanol solutions of 1α,25-dihydroxyvitamin D₃ (the standard substance) and Compounds 14, 16, 19 and 21 were prepared at various concentrations. Bovine thymus 1α,25-dihydroxyvitamin D₃ receptor was purchased from Yamasa Biochemcal (Choshi, Chiba, Japan) (lot. 112631) and, just before use, one ampule (approximately 25 mg) of the receptor was dissolved in 55 mL of 0.05 M phosphate buffer (pH 7.4) containing 0.3M KCl and 5 mM dithiothreitol.

Each of the ethanol solutions (50 μl) of the test compounds and 1α,25-dihydroxyvitamin D₃ was put into a respective tube with 500 μl (0.23 mg protein) of the receptor solution, pre-incubated at 25° C. for 1 hour, and [³H]-1α,25-dihydroxyvitamin D₃ was added at the final concentration of 0.1 nM, followed by incubation overnight at 4° C. Each of the reaction mixtures was mixed with DCC (dextran coated charcoal), left for 30 minutes at 4° C. and centrifuged at 3000 rpm for ten minutes to separate the bound and free forms of [³H]-1α,25-dihydroxyvitamin D₃. Each of the resultant supernatants (500 μl) was mixed with ACS-II (9.5 ml) (AMERSHAM, England) for radioactivity measurement.

The binding properties of the test compounds expressed in relative value with that of 1α,25-dihydroxyvitamin D₃ taken as 100 were shown in Table below. The values were calculated according to the following equation.

X=(y/x)×100

X: relative VDR binding property of a test compound

y: concentration of 1α,25-dihydroxyvitamin D₃ that inhibits 50% of the binding of [³H]-1α,25-dihydroxyvitamin D₃ and VDR

x: concentration of the test compound that inhibits 50% of the binding of [³H]-1α,25-dihydroxyvitamin D₃ and VDR

TABLE

| | Compound | Compound | Compound | Compound |
|---|---|---|---|---|
| Compound | 14 | 16 | 19 | 21 |
| VDR binding | 0.035 | 70 | 0.24 | <0.01 |
| properties | | | | |

### INDUSTRIAL APPLICABILITY

As described above, the vitamin D derivatives of the present invention are novel, exhibit excellent physiological activities, and are expected to be useful as medicines, for example, for calcium metabolism regulation. The compounds of the present invention may be useful as reagents for studying metabolism of active vitamin D₃ (i.e., 1α,25-dihydroxyvitamin D₃).

1. A vitamin D derivative of Formula (1): 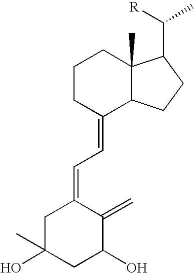
wherein R is straight or branched alkyl optionally substituted with hydroxy.

2. The vitamin D derivative of claim 1 wherein R is straight or branched C₁₋₁₂ alkyl substituted with hydroxy.

3. The vitamin D derivative of claim 1 wherein R is straight or branched C₁₋₁₀ alkyl substituted with hydroxy.

4. The vitamin D derivative of claim 1 wherein R is 4-hydroxy-4-methylpentyl or 4-ethyl-4-hydroxyhexyl.

5. The vitamin D derivative of claim 1 wherein R is 4-hydroxy-4-methylpentyl.
